(19) United States
(12) Patent Application Publication
(10) Pub. No.: US 2025/0288368 A1
(43) Pub. Date: Sep. 18, 2025

(54) RADAR GRAPHICAL USER INTERFACE FOR ROBOTIC MEDICAL SYSTEMS

(71) Applicant: Intuitive Surgical Operations, Inc., Sunnyvale, CA (US)

(72) Inventors: Hong Seo Lim, Sunnyvale, CA (US); Kenneth Whaler, Seaview, WA (US); Anthony M. Jarc, Johns Creek, GA (US); Xi Liu, Peachtree Corners, GA (US)

(73) Assignee: Intuitive Surgical Operations, Inc., Sunnyvale, CA (US)

(21) Appl. No.: 19/080,289

(22) Filed: Mar. 14, 2025

Related U.S. Application Data
(60) Provisional application No. 63/566,074, filed on Mar. 15, 2024.

(51) Int. Cl. A61B 34/00; A61B 34/30; A61B 90/00

(52) U.S. Cl. CPC A61B 34/25; A61B 34/30; A61B 90/361; A61B 2090/373

(57) ABSTRACT

A robotic medical system with a radar chart including performance indicators is described. A system can include one or more processors, coupled with memory, to receive, from a robotic medical system, a data stream of a medical procedure performed by the robotic medical system. The one or more processors can determine, using at least the data stream, metrics corresponding to types, wherein the metrics are indicative of performance of the medical procedure with the robotic medical system. The one or more processors can identify baseline metrics corresponding to the types. The one or more processors can generate data to cause a graphical user interface to display a radar plot comprising the metrics normalized based on the corresponding baseline metrics.

### CROSS-REFERENCE TO RELATED PATENT APPLICATION

This application claims the benefit of, and priority to, under 35 U.S.C. § 119, U.S. Provisional Patent Application No. 63/566,074, filed Mar. 15, 2024, which is hereby incorporated by reference herein in its entirety.

### BACKGROUND

A robotic medical system can include an instrument for performing a medical session or procedure. For example, the instrument can be used to perform surgery, therapy, or a medical evaluation. The robotic medical system can collect videos of the medical procedure.

### SUMMARY

Technical solutions disclosed herein can include a graphical user interface for displaying objective performance indicators (OPIs) of a medical procedure performed by a robotic medical system. For example, this technology can generate a radar chart to display multiple OPIs along with a benchmark range, which allows a user to check multiple OPIs at once, and also easily understand the relative magnitude of the OPIs with reference to a benchmark. The performance indicators can be normalized so that a medical practitioner can understand relative magnitudes between the performance indicators, and the performance indicators can each be plotted on a different axis. The plot can display performance indicators of other medical procedures that form a benchmark. For example, a group of other medical procedures can form the benchmark. This group can be historical medical procedures for a particular hospital, historical medical procedures of a particular practitioner, or historical medical procedures for a group of practitioners. Performance indicators for the group can be generated, and displayed on the plot of the performance of the medical practitioner so that the medical practitioner can view their performance with respect to the group. For example, a range (e.g., a top 75% and a bottom 25%) for each performance indicator for the group can be plotted on the axes to allow the medical practitioner to understand their performance relative to the performance range of groups. In this regard, the plot can provide a compact representation of the performance of the medical practitioner while using a small amount of graphical user interface elements, and therefore cause the computing system to consume less processing resources, memory resources, or power resources. Furthermore, the plot can be displayed along with a video of a medical procedure. The user can view a video of the medical procedure together with the plot. As the user moves between different phases and steps of the medical procedure in the video, the plot can be updated to display performance indicators for the corresponding phase or step that the user has selected.

At least one aspect of the present disclosure is directed to a system. The system can include one or more processors, coupled with memory, to receive, from a robotic medical system, a data stream of a medical procedure performed by the robotic medical system. The one or more processors can determine, using at least the data stream, metrics corresponding to types, wherein the metrics are indicative of performance of the medical procedure with the robotic medical system. The one or more processors can identify baseline metrics corresponding to the types. The one or more processors can generate data to cause a graphical user interface to display a radar plot including the metrics normalized based on the corresponding baseline metrics.

The one or more processors can receive the data stream from the robotic medical system during the medical procedure. The one or more processors can generate the data to cause the graphical user interface to display the radar plot during the medical procedure. The one or more processors can generate an alarm during the medical procedure responsive to at least one of the metrics satisfying a threshold.

The one or more processors can generate the data to cause the graphical user interface to display the radar plot including three or more axes on a single plane to display three or more metrics of the metrics. The three or more axes can extend outwards from a single point representing a first value of the three or more metrics to second points representing second values of the three or more metrics, the second values greater than the first value.

The one or more processors can receive a video of the medical procedure from the robotic medical system. The one or more processors can receive, via the graphical user interface, a selection of a segment of segments of the video of the medical procedure. The one or more processors can generate the data to cause the graphical user interface to display a portion of the video corresponding to the segment and the radar plot to display the metrics corresponding to the segment.

The one or more processors can receive a video of the medical procedure from the robotic medical system. The one or more processors can cause the graphical user interface to include an element to play the video. The one or more processors can cause the graphical user interface to include a timeline including segments. The one or more processors can receive, via the element or the timeline, a scan of the video to a point corresponding to a segment of the segments of the medical procedure. The one or more processors can select the segment from the segments responsive to receiving the scan to the point. The one or more processors can generate the data to cause the graphical user interface to display a portion of the video corresponding to the segment and the radar plot to display the metrics corresponding to the segment.

The one or more processors can receive a selection to display the metrics for an entirety of the medical procedure. The one or more processors can generate the metrics for the entirety of the medical procedure using the data stream. The one or more processors can generate the data to cause the graphical user interface to display the radar plot to display the metrics for the entirety of the medical procedure.

The one or more processors can receive, via the graphical user interface, a selection of a segment of segments of the medical procedure, wherein the segment is a phase of the medical procedure. The phase can include at least one of transection of an anatomical structure of a patient, extraction of the anatomical structure, reconstruction of the anatomical structure after opening the anatomical structure, dissection of the anatomical structure, or exposure of the anatomical structure. The one or more processors can generate the data to cause the graphical user interface to display the radar plot to display the metrics for the segment.

The one or more processors can receive, via the graphical user interface, a selection of a step of steps of a particular phase of phases of the medical procedure. The one or more processors can generate the data to cause the graphical user interface to display the radar plot to display the metrics for the step.

The one or more processors can generate, using at least the data stream at least one metric. The metrics can include at least one of an amount of energy consumed by the robotic medical system, a total duration of a segment of the medical procedure, a total linear distance an instrument of the robotic medical system travelled during the segment, a total angular distance the instrument of the robotic medical system travelled during the segment, a total number of operations of a clutch or brake for the instrument of the robotic medical system, or a total number of operations of a clutch or brake for an endoscope of the robotic medical system.

The one or more processors can cause the graphical user interface to plot points on three or more axes of the radar plot based on values of the metrics. The one or more processors can cause the graphical user interface to plot segments between the points on neighboring axes of the three or more axes.

The one or more processors can receive data streams for medical procedures. The one or more processors can generate the metrics for the medical procedures. The one or more processors can determine at least one percentile of the metrics. The one or more processors can generate the data to cause the graphical user interface to display the radar plot including three or more axes on a single plane to display three or more metrics of the metrics and the at least one percentile of the three or more metrics.

The one or more processors can receive a first data streams for a first medical procedures performed by a first medical practitioner with the robotic medical system. The one or more processors can generate, using at least the first data streams, first metrics for the first medical practitioner. The one or more processors can receive a second data streams for a second medical procedures performed by a second medical practitioner with the robotic medical system. The one or more processors can generate, using at least the second data streams, second metrics for the first medical practitioner. The one or more processors can generate the data to cause the graphical user interface to include a first radar plot to display the first metrics for the first medical practitioner and a second radar plot to display the second metrics for the second medical practitioner.

The one or more processors can receive, via the graphical user interface, a shape drawn on the radar plot, the shape identifying points on three or more axes of the radar plot. The one or more processors can detect that a value of one metric of the metrics is greater than or less than a value corresponding to one point of the points drawn on an axis of the one metric. The one or more processors can generate an alarm responsive to a detection that the value of the one metric is greater than or less than the value corresponding to the one point.

The one or more processors can receive, via the graphical user interface, a shape drawn on the radar plot, the shape defined by points on three or more axes of the radar plot. The one or more processors can search medical procedures using the shape drawn on the radar plot to identify a second medical procedure associated with the metrics that conforms to the shape. The one or more processors can cause the graphical user interface to display a video of the second medical procedure.

At least one aspect of the present disclosure is directed to a method. The method can include receiving, one or more processors, coupled with memory, from a robotic medical system, a data stream of a medical procedure performed by the robotic medical system. The method can include determining, by the one or more processors, using at least the data stream, metrics corresponding to types, wherein the metrics are indicative of performance of the medical procedure with the robotic medical system. The method can include identifying, by the one or more processors, baseline metrics corresponding to the types. The method can include generating, by the one or more processors, data to cause a graphical user interface to display a radar plot including the metrics normalized based on the corresponding baseline metrics.

The method can include receiving, by the one or more processors, the data stream from the robotic medical system during the medical procedure. The method can include generating, by the one or more processors, the data to cause the graphical user interface to display the radar plot during the medical procedure. The method can include generating, by the one or more processors, an alarm during the medical procedure responsive to at least one of the metrics satisfying a threshold.

The method can include receiving, by the one or more processors, a video of the medical procedure from the robotic medical system. The method can include causing, by the one or more processors, the graphical user interface to include an element to play the video. The method can include causing, by the one or more processors, the graphical user interface to include a timeline including segments. The method can include receiving, by the one or more processors, via the element or the timeline, a scan of the video to a point corresponding to a segment of the segments of the medical procedure. The method can include selecting, by the one or more processors, the segment from the segments responsive to receiving the scan to the point. The method can include generating, by the one or more processors, the data to cause the graphical user interface to display a portion of the video corresponding to the segment and the radar plot to display the metrics corresponding to the segment.

The method can include receiving, by the one or more processors, a selection to display the metrics for an entirety of the medical procedure. The method can include generating, by the one or more processors, the metrics for the entirety of the medical procedure using the data stream. The method can include generating, by the one or more processors the data to cause the graphical user interface to display the radar plot to display the metrics for the entirety of the medical procedure.

At least one aspect of the present disclosure is one or more storage media storing instructions thereon, that, when executed by one or more processors, cause the one or more processors to receive, from a robotic medical system, a data stream of a medical procedure performed by the robotic medical system. The instructions can cause the one or more processors to determine, using at least the data stream, metrics corresponding to types, wherein the metrics are indicative of performance of the medical procedure with the robotic medical system. The instructions can cause the one or more processors to identify baseline metrics corresponding to the types. The instructions can cause the one or more processors to generate data to cause a graphical user interface to display a radar plot including the metrics normalized based on the corresponding baseline metrics.

The instructions can cause the one or more processors to receive a video of the medical procedure from the robotic medical system. The instructions can cause the one or more processors to cause the graphical user interface to include an element to play the video. The instructions can cause the one or more processors to cause the graphical user interface to include a timeline including segments. The instructions can cause the one or more processors to receive via the element or the timeline, a scan of the video to a point corresponding to a segment of the segments of the medical procedure. The instructions can cause the one or more processors to select the segment from the segments responsive to receiving the scan to the point. The instructions can cause the one or more processors to generate the data to cause the graphical user interface to display a portion of the video corresponding to the segment and the radar plot to display the metrics corresponding to the segment.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 depicts an example computing system to generate a radar chart including performance indicators of a robotic medical system.

FIG. 2 depicts a graphical user interface including a radar chart including metrics of a robotic medical system.

FIGS. 3A-B depicts a graphical user interface including radar charts that correspond to segments of a video of a medical procedure.

FIG. 4 depicts a graphical user interface including comparisons between early and late stage cases.

FIG. 5 depicts a graphical user interface including radar charts of performance indicators of different medical practitioners.

FIG. 6 depicts a method of generating a radar chart including performance indicators of a robotic medical system.

FIG. 7 depicts an example computing architecture of a data processing system.

### DETAILED DESCRIPTION

Following below are more detailed descriptions of various concepts related to, and implementations of, methods, apparatuses, and systems for a radar graphical user interface for a robotic medical system. The various concepts introduced above and discussed in greater detail below may be implemented in any of numerous ways.

This disclosure is generally directed to displaying performance information of a medical procedure performed by a robotic medical system by a medical practitioner, such as a surgeon, a doctor, a nurse, a technician, or another user. A computing system can collect videos or frames of a medical procedure performed by a medical robotic system, such as a surgery, a therapy, or a treatment. Furthermore, the computing system can collect kinematics information describing the operation, control, or performance of the medical robotics system (e.g., energy consumed by the medical robotic system or instrument manipulators, movement distances of instrument manipulators, numbers of clutches of an instrument manipulator to float joints of the instrument manipulator, etc.). The computing system can generate multiple performance indicators to quantify the performance of the surgical procedure. However, each performance indicator can have a different scale or range. Furthermore, a medical practitioner can have difficulty reviewing and understanding multiple performance indicators simultaneously in a quick and efficient manner. For example, surgeons may want to compare their performance indicators against other surgeons, e.g., expert surgeons or experienced surgeons. However, to simultaneously compare multiple performance indicators of multiple surgeons may be difficult and time consuming for the surgeon.

Furthermore, the surgeon may be interested in their performance compared to other surgeons in real-time, or during a particular segment or phase of a recorded surgical procedure. Without a compact representation of the multiple performance indicators for the one surgeon compared to multiple other surgeons, a significant amount of resources for a graphical user interface can be consumed, e.g., a large amount of area in a graphical user interface can be consumed or cluttered with various representations of performance indicators or graphics, and therefore a significant amount of memory resources may be needed to store the various graphic elements and a significant amount of processing resources may be used to render the graphic elements. Thus, due to the numerous performance indicators generated for a medical procedure performed by robotic medical system, it can be technically challenging for a computing system to display the numerous performance indicators in an efficient and accurate manner without consuming excess computing resources or graphical user interface resources. This can result in the inability to efficiently provide timely, useful, and accurate performance feedback on the operation of the robotic medical system, thereby impacting the performance or outcome of the medical procedure performed by the medical robotic system or subsequent medical procedures performed by robotic medical systems.

To solve these, and other technical problems, technical solutions of this disclosure can include generating an improved graphical user interface for displaying OPIs of a medical procedure performed by a robotic medical system. In particular, this technology can generate a radar chart to display multiple OPIs along with a benchmark range, which allows a user to check multiple OPIs at once, and also easily understand the relative magnitude of the OPIs with reference to a benchmark. Further, this technology can generate a shape-based OPI based on a combination of OPIs displayed in the radar graphical user interface. The shape-based OPI can represent a signature or fingerprint of performance of the medical procedure, which this technology can utilize to provide alerts or other feedback to manage, improve, or otherwise control the performance of the medical procedure or subsequent medical procedures performed by the robotic medical system.

For example, the radar chart can display three or more axes on a single plane for performance indicators of a medical procedure performed by a medical robotic system. The plot can be a two-dimensional plot or have multiple axes in a single plane. The plot can be a radar plot, in some examples. The plot can provide a compact view of multiple performance indicators (such as normalized performance indicators) along the various axes. The performance indicators can be normalized so that a medical practitioner can understand relative magnitudes between the performance indicators, and the performance indicators can each be plotted on a different axis. The points plotted along each axis can be connected or joined together by segments or lines. For example, segments or lines can be drawn between points plotted on neighboring axis. In this regard, a shape can be drawn on the plot that represents a practitioner's performance. Furthermore, the plot can display performance indicators of other medical procedures that form a benchmark. For example, a group of other medical procedures can form the benchmark. This group can be historical medical procedures for a particular hospital, historical medical procedures of a particular practitioner, or historical medical procedures for a group of practitioners, etc. Performance indicators for the group can be generated, and displayed on the plot of the performance of the medical practitioner so that the medical practitioner can view their performance with respect to the group. For example, a range (e.g., a top 75% and a bottom 25%) for each performance indicator for the group can be plotted on the axes to allow the medical practitioner to understand their performance relative to the performance range of groups.

In this regard, the plot can provide a compact representation of the performance of the medical practitioner while using a small amount of graphical user interface elements, and therefore cause the computing system to consume less processing resources, memory resources, or power resources. Furthermore, the plot can be displayed along with a video of a medical procedure. The user can view a video of the medical procedure together with the plot. As the user moves between different phases or steps of the medical procedure in the video, the plot can be updated to display performance indicators for the corresponding phase or step that the user has selected. In this regard, the plot can be sequenced with the video, to allow the medical practitioner to understand the performance of the medical procedure during each phase or step of the medical procedure. By sequencing the video of the medical procedure and the plot together, a medical practitioner can have a compact graphical user interface that can be easy to navigate while providing granular detail in a holistic manner.

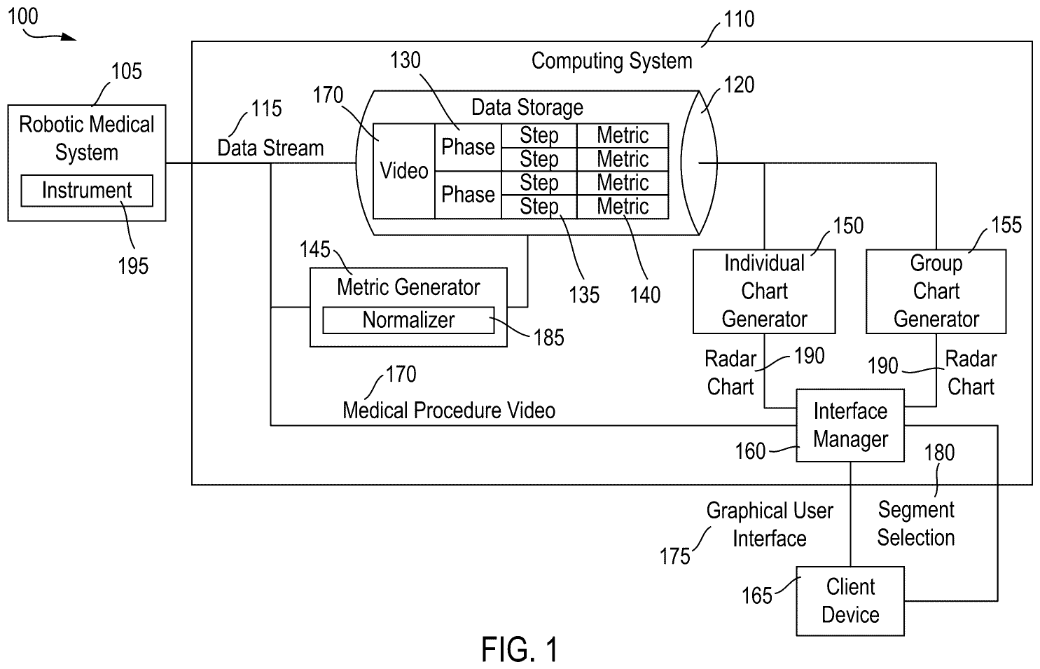

Referring now to FIG. 1, among others, an example system 100 including a computing system 110 to generate a radar GUI 190 (or radar graphical user interface) including performance indicators of a robotic medical system 105 is shown. The example computing system 110 can be a data processing system, a computer, a desktop computer, a control system, a console system, an embedded system, a cloud computing system, or any other type of computing system. The computing system 110 can be an on-premises system or an off-premises system. The computing system 110 can be a hybrid system, where some components of the computing system 110 are located on-premises, and some components of the computing system 110 are located off-premises.

The computing system 110 can receive a data stream 115 from a robotic medical system 105. The robotic medical system 105 can be a robotic system, apparatus, or assembly including at least one instrument 195. The instrument 195 can be or include a tip or end. The tip or end can be installed with or to the instrument 195. The tip can be a removable or permanent component of the instrument 195 or the robotic medical system 105. For example, the tip can be a scalpel, a scissors, a monopolar curved scissors (MCS), a cautery hook tip, a cautery spatula tip, a needle driver, forceps, a round tooth retractor, a drill, or a clip applier. The instrument 195 can be or include a robotic arm, a robotic appendage, a robotic snake, or any other motor controlled member that can be articulated by the robotic medical system. The instrument 195 can include at least one actuator, such as a motor, servo, or other device. The instrument 195 can be manipulated by motors, servos, actuators, or other devices to perform a medical procedure. The robotic medical system 105 can perform a medical session or medical procedure. For example, the robotic medical system 105 can articulate the instrument to perform surgery, therapy, or a medical evaluation with the instrument 195. A medical practitioner, such as a surgeon, technician, nurse, or other operator can provide input via a user device or input apparatus to manipulate the instrument 195 to perform a medical procedure. The robotic medical system 105 can include an endoscope, in some implementations. The endoscope can be an instrument 195 that is manipulated by the medical practitioner and controlled via a motor, servo, or other input device.

The robotic medical system 105 can produce a data stream 115. The data stream 115 can be data recorded, collected, or generated by the robotic medical system 105. The data stream 115 can be video data, image data, frame data, or other image information collected by an endoscope of the robotic medical system 105. For example, the data stream 115 can be or include a medical procedure video 170. The medical procedure video 170 can be a video or video stream generated by the endoscope of the robotic medical system 105. The medical procedure video 170 can be a video of the medical procedure performed by the robotic medical system 105. The medical procedure video 170 can include frames or images that track the instrument 195 within a field of view of a camera of the endoscope.

The data stream 115 can include motor data, kinematics data, power consumption data, or other information describing the operation of the robotic medical system 105. For example, the data stream 115 can indicate or track the motions of the instruments 195. For example, the data stream 115 can indicate or track turns or rotations of the instruments 195. For example, the data stream 115 can indicate or track angular or linear distances traveled by the instrument 195 during the medical procedure. For example, the data stream 115 can indicate the type of the medical procedure. For example, the data stream 115 can indicate different phases or steps of the medical procedure.

The computing system 110 can receive a data stream 115 from the robotic medical system 105. The data stream 115 can be data messages, data packets, signals, a video stream, telemetry, or messages. The computing system 110 can store at least a portion of the data stream 115 in a data storage 120. The data storage 120 can be a server system, a database, a hard drive, or set of hard drives. The data storage 120 can store the video 170, or a reference to the video 170. The data storage 120 can include indications or tags of the various segments or portions of the video 170. The segments can be phases 130 or steps 135 of the medical procedure that the video 170 is captured for. The phases 130 can indicate various high level sections of the video 170 or of the medical procedure. A medical procedure can include multiple phases 130, while at least some of the multiple phases 130 can include multiple steps 135.

The phases 130 can include transection of an anatomical structure of a patient. For example, the phase 130 can include transecting or dividing an anatomical structure, e.g., opening the anatomical structure. Transection can include cutting transversely across an anatomical structure. The phase 130 can include extraction of an anatomical structure. For example, extraction can include cutting out or removing an anatomical structure from a patient or from an area or cavity of a patient. The phase 130 can include reconstruction of an anatomical structure. For example, after opening, cutting, or exposing an anatomical structure, the robotic medical system 105 can reconstruct the anatomical structure. For example, the robotic medical system 105 can stitch up an opening in an anatomical structure, staple closed an opening in an anatomical structure, or otherwise reconstruct an anatomical structure after opening or cutting the structure. The phase 130 can include dissection, such as cutting or dissecting an anatomical structure or patient. The phase 130 can include exposure of an anatomical structure. For example, the phase 130 can include peeling back tissue, cutting back tissue, moving other anatomical structures, or removing bones to expose or access an anatomical structure. The phases 130 can include individual steps 135. The steps 135 collectively can form or complete a particular phase 130. The steps 135 can include steps for a cholecystectomy, for example specimen removal, litigation or division of a cystic artery, dissection of a gallbladder of a liver off a liver bed, etc. The steps 135 can be steps of any type of medical procedure. The steps 135 can be specific to a procedure type. The steps 135 can be procedure type specific, while the phases 130 can be universal or apply to multiple procedure types.

The computing system 110 can execute at least one model trained by machine learning to identify or detect each phase 130 or step 135 in the video 170, and can label segments of time in the video 170 with an indication of a type or name of the phase 130 or step 135. The model can be an image transformer, a convolutional neural network, or any other model that can classify the phases 130 or steps 135 in the video 170. The model can be trained or learn to detect and segment the video 170 into phases 130 and steps 135.

The computing system 110 can include at least one metric generator 145. The metric generator 145 can generate the metrics 140. The metric generator 145 can store the metrics 140 in the data storage 120. The metric generator 145 can generate metrics for each phase 130 or step 135. The metric generator 145 can generate the metrics 140 for the video 170, the phases 130, or the steps 135 responsive to the model executing to segment the video 170 into the phases 130 and steps 135. The metric generator 145 can generate the metrics 140 using at least a portion of the data stream 115. The metric generator 145 can generate the metrics 140 based on the data stream 115. The metric generator 145 can generate the metrics 140 for segments of the video 170. For example, the metric generator 145 can generate the metrics 140 for particular sections or windows of the video 170, e.g., the metric 140 can be a metric associated with a first timestamp of the video 170 and a second timestamp of the video 170. The metrics 140 can be generated with a portion of the data stream 115 corresponding to the particular segment of the video 170.

The metric generator 145 can generate metrics 140 for a smallest segment of the video 170, such as a step 135, and then use the metrics 140 to generate metrics 140 for larger segments of the video 170. For example, metrics 140 generated for a sub-segments can be aggregated into a metric 140 for the segment that the sub-segments make up. With the metrics generated for the steps 135, the metric generator 145 can generate a metric for a phase 130 that includes multiple steps 135. For example, the metric generator 145 can aggregate metrics 140 for steps 135 corresponding to a particular phase 130 into a metric 140 for the phase.

The metrics 140 can each be generated for a different type or metric type. For example, the metric generator 145 can generate the metrics 140 to correspond to multiple types. The metrics 140 and metric types can indicate or be indicative of performance of the robotic medical system performing the medical procedure. The metrics 140 and metric types can indicate or be indicative of performance of the medical procedure. The metrics 140 and metric types can indicate or be indicative of performance of an operator of the robotic medical system.

The various metric types of metrics can be a metric 140 indicating an amount of energy or power consumed by the robotic medical system 105. The various metric types of metrics can be a metric 140 indicating an amount of energy or power consumed by the robotic medical system 105 to operate an individual instrument 195 or endoscope. The various metric types of metrics can be a metric 140 indicating total duration of a segment of the medical procedure. For example, the metric 140 can be a length of time of the particular segment of the video 170, such as a length of time of the step 135, the phase 130, or the entire medical procedure. The various metric types of metrics can be a metric 140 indicating a total linear distance the instrument 195 of the robotic medical system 105 travelled during the segment. For example, the metric 140 can indicate a total distance that the instrument 195 traveled during the steps 135, the phases 130, or the entire video 170. The various metric types of metrics can be a metric 140 indicating a total angular distance of the instrument 195 of the robotic medical system 105 during the segment. The angular distance can indicate an amount or distance that the instrument 195 is rotated during manipulations. The metric 140 can indicate a total angular distance that the instrument 195 traveled during the steps 135, the phases 130, or the entire video 170.

The various metric types of metrics can be a metric 140 indicating a total number of operations or a clutch or brake. For example, the metric 140 can indicate a total number of actuators or activations of a clutch or brake for the instrument 195 or the endoscope of the robotic medical system 105. The clutch or brake can be operated to float joints of the instrument 195 or endoscope. The metric 140 can indicate a total number of operations or a brake or clutch during the steps 135, the phases 130, or the entire video 170.

The metric generator 145 can include at least one normalizer 185. The normalizer 185 can determine, generate, or identify baseline metrics. The baseline metrics can be for various metric types. For example, the baseline metrics can correspond to the types of the metrics 140. The baseline metrics can indicate historical ranges, e.g., maximums, minimums, averages, medians, etc. The normalizer 185 can normalize each metric 140 with the baseline metrics for the corresponding type of the metric 140. In this regard, each metric 140 can be generated and normalized so that each metric 140 is on the same scale. The scale can be 0-10, 0-100, 0-5, or any other scale. By normalizing the metrics 140, the metrics displayed in radar charts 190 can be uniform and understandable for a user. Even though each type of metric 140 may have a different range, normalizing the metrics 140 can allow for the metrics to be simultaneously displayed without confusing a user who views the radar charts 190. The metrics 140 can all be normalized or scaled to share a common range.

The computing system 110 can generate data to cause a graphical user interface 175 to include or display a radar GUI 190. The computing system 110 can generate a plot 190 to include three or more axes for performance indicators or metrics 140 of the medical session and the operation of a medical robotic system on a single plane (e.g., in two-dimensions or on a radar plot). The radar GUI 190 can include the metrics 140. The radar GUI 190 can include the metrics 140 normalized based on corresponding baseline metrics determined by the normalizer 185. The radar GUI 190 can be a two-dimensional chart where three or more axes are included on a single plane. Each axes can display values of a different metric 140 of a different type. The chart 190 can include one axis, two axes, three axes, or any other number of axes. Each axes can display a different type of metric 140, e.g., energy, pedal count, endoscope clutch count, duration, total instrument path length, total instrument angular path length, hand controller clutch count, or any other metric type indicating performance of the robotic medical system 105.

For example, a user can simultaneously or concurrently view different normalized metrics 140 on a single radar GUI 190, without needing the user to view multiple charts and understand the relative differences in ranges of the different metrics 140. The radar GUI 190 can include three or more axes that extend outwards away from a point or center. The center can represent a lowest or zero point for each axes. The axes can extend away from the center outwards to an end. A first end of each axes can start at the center, and extend outwards to a second end. Each axes can be spaced in equal or unequal angles between axes. The second end of each axes can represent a second point or value greater than a value represented by the first point or center point. For example, the three or more axes can extend outwards from a single point representing a first value of the three or more metrics to second points representing second values of the three or more metrics, where the second values greater than the first value.

The radar charts 190 can be generated for an entire medical procedure. The radar charts 190 can be generate for specific segments of the medical procedure or the video 170. For example, the radar GUI 190 can be generated for a particular step 135 or phase 130 of the video 170. For example, the computing system 110 can include an interface manager 160. The interface manager 160 can receive a segment selection 180. The segment selection 180 can be a user input by a user via a client device 165 indicating that the radar GUI 190 should be updated or displayed with metrics 140 corresponding to the particular segment selected by the user. Responsive to the segment selection 180, the computing system 110 can update the radar GUI 190 to display metrics 140 for the selected segment. For example, if the user selects a particular step 135 or phase 130, the radar GUI 190 can be updated to display metrics for the corresponding step 135 or phase 130. In some implementations, the graphical user interface 175 plays the medical procedure video 170. As the video moves to different phases 130 or steps 135, the radar GUI 190 can be updated by the computing system 110 to display the radar GUI 190 with metrics corresponding to the phase 130 or step 135 currently playing in the video 170.

In some implementations, the user, via the client device 165, can select to view metrics 140 for an entirety of a medical procedure. In this regard, the computing system 110 can generate metrics 140 for the entire medical procedure. For example, the computing system 110 can aggregate the metrics 140 for the various steps 135 or phases 130 together to generate a single set of metrics 140 for various metric types for the entire medical procedure. The computing system 110 can generate data to cause the graphical user interface 175 to display a radar GUI 190 with metrics 140 for the entire medical procedure or medical procedure video 170.

The computing system 110 can include an individual chart generator 150. The individual chart generator 150 can generate a radar GUI 190 for an individual operator of the robotic medical system 105. For example, the radar GUI 190 can be generated with historical metrics 140 of the operator. The radar GUI 190 can be generated to display metrics 140 for an individual video 170 which are normalized against baseline metrics 140 determined from past medical procedures performed by the operator with the robotic medical system 105. The individual chart generator 150 can identify percentiles for the various types of metrics 140 for the operator. The percentiles can indicate a seventy fifth percentile, a fiftieth percentile, or a twenty fifth percentile, etc. for the operator. The individual chart generator 150 can generate the radar GUI 190 to overlay metric percentiles for past medical procedures with current metrics 140 for the medical procedure on the radar GUI 190.

The computing system 110 can include at least one group chart generator 155. The group chart generator 155 can generate the radar GUI 190 to display percentiles for a group of operators. For example, the group chart generator 155 can generate percentiles for a group of operators for each metric type. The group chart generator 155 can generate a radar GUI 190 that overlays metrics 140 of an individual operator with percentiles of the metric 140 for the group of operators. In this regard, the radar GUI 190 generated by the group chart generator 155 can generate a radar GUI 190 that allows a user to compare their own metrics 140 for a particular medical procedure against the metrics 140 of a group of operators. The group of operators can be a high performing selected group of operators (e.g., the operators can be associated with metrics 140 greater than a threshold).

The interface manager 160 can receive a window of time selected via the client device 165 and the graphical user interface 175. The graphical user interface 175 can include a date range allowing a medical practitioner to scan through different groups of medical procedures based on date, e.g., select a date range. The window can be a date range, a month range, or a year range. The individual chart generator 150 and the group chart generator 155 can generate the radar charts 190 with data corresponding to the window. The individual chart generator 150 and the group chart generator 155 can generate the radar charts 190 based on data that is tagged with a timestamp within the window. For example, the percentiles displayed in the radar charts 190 can be generated from data that includes a timestamp within the window. The computing system 110 can generate the radar GUI 190 based on data of the medical procedures falling within the selected date range.

The computing system 110 can include at least one interface manager 160. The interface manager 160 can generate at least one graphical user interface 175. The interface manager 160 can generate data to cause the client device 165 to display the graphical user interface 175. The interface manager 160 can generate data to cause the graphical user interface 175 to include at least one radar chart or GUI 190. The interface manager 160 can generate data to cause the graphical user interface 175 including the radar GUI 190 to be rendered, displayed, or provided on an interface of the client device 165. The interface manager 160 can receive input from the client device 165. For example, the interface manager 160 can receive the segment selection 180 from the client device 165 via the graphical user interface 175. The interface manager 160 can receive alarm thresholds for the metrics 140 from the client device 165 via the graphical user interface 175. The client device 165 can be a smartphone, a laptop computer, a desktop computer, a console, a component of the robotic medical system 105, a human machine interface, or a server system that provides graphical user interfaces 175 to user devices. The client device 165 can include a display, such as a light emitting display (LED), an organic light emitting display (OLED), or a liquid crystal display (LCD). The client device 165 can receive input via input devices, such as a touch screen display (e.g., capacitive touchscreens or resistive touchscreens), a keyboard, a mouse, a joystick, a manipulator, or a microphone.

In some implementations, the computing system 110 can identify outcomes of medical procedures. For example, the outcomes can indicate that the medical procedure was successful in accomplishing an objective or unsuccessful in accomplishing an objective. For example, the outcome may indicate that the medical procedure was successful in removing a tumor, stopping internal bleeding, performing a bypass, or any other procedure outcome. The computing system 110 can generate a radar chart that displays metrics 140 for an operator associated with successful or unsuccessful outcomes. For example, the computing system 110 can generate a radar GUI 190 to include metrics 140 associated with successful medical procedures. The computing system 110 can generate a radar GUI 190 to include metrics 140 associated with unsuccessful medical procedures. The computing system 110 can generate the radar GUI 190 to indicate percentiles for metrics 140 of successful medical procedures or to indicate percentiles for metrics 140 of unsuccessful medical procedures. The computing system 110 can generate separate radar charts 190 to represent metrics 140 for successful and unsuccessful medical procedures. The computing system 110 can plot the metrics 140 for an operator of the robotic medical system 105 for a particular medical procedure with percentiles of metrics 140 for successful or unsuccessful medical procedures. This can allow a medical practitioner to understand how their performance conforms to performance indicators of successful and unsuccessful medical procedures.

The computing system 110 can generate the radar plot 190 intraoperatively or during the medical procedure. The computing system 110 can collect the data stream 115 of the medical procedure intraoperatively or in real-time. The computing system 110 can generate a real-time dynamic radar GUI 190 that tracks the performance of the medical procedure performed by the operator. The computing system 110 can receive the data stream 115 intraoperatively, or during the medical procedure. The computing system 110 can receive the data stream 115 in real-time, or as the data of the data stream 115 is generated, collected, or measured by the robotic medical system 105. The computing system 110 can use the data stream 115 to generate the radar GUI 190 intraoperatively, or during the medical procedure as the data stream 115 is received. As new data of the data stream 115 is received, the computing system 110 can regenerate or update the metrics 140 displayed in the radar GUI 190. In this regard, the computing system 110 can provide an operator of the robotic medical system 105 real-time feedback on their performance in operating the robotic medical system 105 to perform the medical procedure.

The computing system 110 can generate at least one alarm during the medical procedure. The computing system 110 can generate at least one alarm responsive to at least one of the metrics 140 satisfying a threshold, e.g., exceeding a threshold or falling below a threshold. For example, the interface manager 160 can cause an alert, pop-up message, or indicator to be displayed in the graphical user interface 175 to represent the alarm responsive to the alarm being generated or raised by the computing system 110. The computing system 110 can generate metrics 140 corresponding to the different types during the medical procedure, and compare each metric 140 to a threshold corresponding to the metric type of the metric 140. In this regard, the computing system 110 can store multiple thresholds, at least one threshold for each of the metric types. For example, a first metric type can have a first threshold or first set of thresholds, and a second metric type can have a second threshold or second set of thresholds. The thresholds can be upper limits for the metrics 140 or lower limits for the metrics 140. The thresholds can be rates of change, e.g., a maximum rate of change for a metric 140. The computing system 110 can track the metrics 140 or the rates of change of the metrics 140 during the medical procedure, and raise alarms during the medical procedure to notify the operator of the robotic medical system 105 of issues. For example, if a surgeon loses sight of their non-dominant hand becoming erratic from fatigue or too much force being applied to tissue causing trauma to the tissue, the computing system 110 can raise an alert, the radar GUI 190 can provide a birds eye or holistic view of performance during a medical procedure.

The thresholds that the computing system 110 uses can be based on user input provided by a user via the client device 165. A user can provide an input to identify a threshold, or identifying a range that that a metric 140 should ideally fall within (e.g., an upper threshold and a lower threshold). In some implementations, a user, via the client device 165, can draw a shape on the radar GUI 190 that identifies points on each axes of the radar GUI 190. The shape can be drawn directly onto the radar GUI 190 by a medical practitioner. The points identified by the shape can be intersections between the shape and the axes of the radar GUI 190. The user can draw multiple shapes to select multiple thresholds. For example, a first shape can identify at least on upper threshold for a type of the metrics 140. A second shape can identify at least one lower threshold for a type of the metrics 140. Based on the shapes, the shapes can identify both upper and lower thresholds. In some implementations, the shape is a target shape or goal for the metrics 140 during a medical procedure. The target shape can, in some implementations, be automatically set or selected by the computing system 110. For example, the computing system 110 can set or select the shape based on the type of the medical procedure, a difficulty of the medical procedure, or average outcomes of the medical procedure. The computing system 110 can track the progress of the real-time shape formed by the metrics 140 plotted on the radar GUI 190 and the target shape or target shape range that the medical practitioner drew. The computing system 110 can store the thresholds and use the stored thresholds to raise alarms (e.g., in real-time or intra-operatively). The computing system 110 can display the shape drawn by the user or the range of values for the metrics 140 on the radar charts 190 with real-time metrics 140 generated for the current medical procedure overlaid onto the range identified. Intraoperatively, the computing system 110 can detect the real-time metrics 140 increasing above an upper threshold or falling below a lower threshold, and generate an alarm or alert to notify the operator of the robotic medical system 105. Based on the thresholds, the computing system 110 can provide an alert, notification, warning, or automatically control or operate the robotic medical system 105 based on a comparison of a current shape of the metrics of the radar GUI 190 with the boundaries or thresholds of the shape drawn in the radar GUI 190 in order to keep the current metrics within a desired shape. The shape boundaries set can indicate good or bad performance of the medical procedure. In some implementations, ideal shapes can be set for different phases or steps of a medical procedure, and the computing system 110 can use the ideal shape to measure performance of the medical procedure for the corresponding current phase or step of the medical procedure.

The computing system 110 can generate the radar GUI 190 to include rings. The rings can be concentric rings or circles centered on the origin of the radar GUI 190 and increasing in diameter outwards from the origin. The rings can be interactive. For example, a user can provide an input to activate various rings as thresholds for the metrics 140. The user can activate the rings as either upper thresholds or lower thresholds. If the user activates a ring as an upper threshold, the computing system 110 can generate an alarm responsive to the metrics 140 exceeding the upper threshold. If the user activates a ring as a lower threshold, the computing system 110 can generate an alarm responsive to the metrics 140 falling below the lower threshold.

The computing system 110 can perform a search of the videos 170 based on a user input via the radar GUI 190. For example, the computing system 110 can receive a user input defining a query based on values for the various types of metrics 140. For example, the computing system 110 can allow a user to draw a shape on a radar plot 190, and use the drawn shape to search against other shapes of other medical procedures to retrieve videos 170 of medical procedures with shapes corresponding to the search shape. For example, a user can draw a shape on the axes of the radar GUI 190 that defines values for the various types of metrics 140. The shape drawn by the operator can define the various metric values. The computing system 110 can search the videos 170 with the shape to identify videos 170 with metrics 140 that correspond to the shape provided via the client device 165. The computing system 110 can retrieve result videos 170 that include metrics 140 that are within a range or within a deviation from the input search query. The computing system 110 can provide the result videos 170 to the operator via the graphical user interface 175. The computing system 110 can apply search filters with a defined radar chart shape to filter down medical procedures based on surgeon skill level or case complexity, both of which can impact the shape of the radar chart.

In some implementations, the computing system 110 can execute at least one model trained by machine learning on the radar GUI 190, or the data displayed in the radar GUI 190. For example, the model can be a neural network, such as a transformer, a convolutional neural network, a feedforward network, or any other type of machine learning model that can learn based on the shape of the metrics 140 plotted in the radar GUI 190, e.g., numeric values of the metrics 140, area of the shape plotted in the radar GUI 190, length of segments or edges of the shape plotted in the radar chart, or the rate of change of the metrics 140 or shape formed by the metrics during different segments of the medical procedure. The rate of change of an area of a shape can provide a metric that indicates whether the performance of a medical procedure is good or bad. The shape can be a signature or fingerprint formed by multiple metrics on the radar GUI 190. The models can be trained on a set of different shapes of metrics, each shape related or linked to a different medical procedure outcome or classification (e.g., a score indicating good or bad performance, a Boolean indicator indicating successful or unsuccessful medical procedures, etc.). The models can be deployed for intra-operative execution by the computing system 110 or post-operative execution to classify shapes, and provide alerts or warnings to indicating good or bad performance of the medical procedure. In this regard, the shape of the metrics itself in the radar GUI 190 can be its own metric that can be used to generate improved alerts or warnings to improve performance of a medical procedure.

The model can output a classification, e.g., ideal performance, unideal performance, a positive indication, a negative indication, an indication not to raise an alarm, or an indication to raise an alarm. The model can classify the shapes of the metrics 140 plotted in the radar GUI 190 as good shapes or bad shapes, shapes that are associated with successful medical procedures or shapes that are associated with unsuccessful medical procedures. For example, the model can determine or predict a performance of the medical procedure based on a shape, signature, or fingerprint formed from multiple metrics on the radar GUI 190. In some implementations, the model can output performance indicators of the medical procedure in real-time or intra-operatively based on the shape of the metrics in the radar GUI 190. The model can identify a high or highest variability metric 140 or set of metrics 140 over multiple medical sessions for a particular medical practitioner. The computing system 110 can generate a notification or message identifying the metrics 140 including variability over a level or threshold.

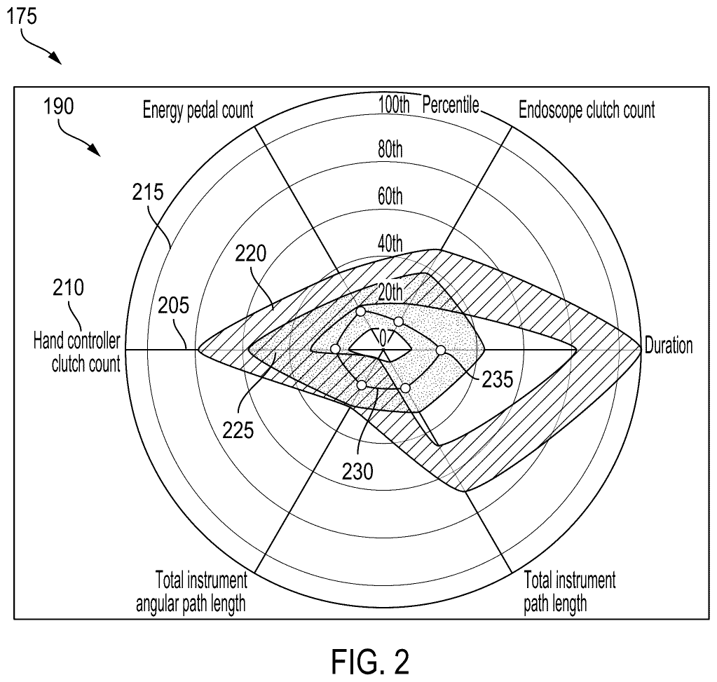

Referring now to FIG. 2, among others, a graphical user interface 175 including a radar GUI 190 including metrics 140 of a robotic medical system 105 is shown. The computing system 110 can generate and provide the GUI 175. The radar GUI 190 can include lines, spokes, or axes 205. The radar GUI 190 can include one axis 205, two axes 205, three axes 205, or any number of axes 205. For example, the radar GUI 190 can include one or more axes 205, two or more axes 205, three or more axes 205, four or more axes 205, five or more axes 205, etc. Each axes 205 can be separated by a particular angle. The angle separating each consecutive axis 205 can be equal. The angle can be three hundred and sixty degrees divided by the total number of axes 205 in the radar GUI 190.

The axes 205 can extend outwards from a first point, origin, or central point of the radar GUI 190. The central point can indicate a minimum value for the metrics 140. The axes 205 can extend from a first point to a second point, where the second point represents a value of the metrics 140 greater than the value of the metrics 140 represented by the first point. The range of the axes 205 can be percentiles, e.g., from a zero percentile to a hundredth percentile. The range of the axes 205 can extend from zero percent to one hundred percent. Each axes 205 can represent a different metric type of the metrics 140. For example, the axes 205 can represent metrics such as energy pedal count, endoscope clutch count, duration, total instrument path length, total instrument angular path length, or hand controller clutch count. Each axes 205 can include a label 210 identifying the metric type. Each axes 205 can display a value of a separate type of metric 140.

The radar GUI 190 can include rings 215. The rings 215 can be concentric circles that are centered about the origin or middle of the radar GUI 190. The rings 215 can each indicate a particular percentile or percentage of the axes 205. The rings 215 can increase in diameter by a constant amount. The rings 215 can represent percentiles or percentages in a constant increasing amount, e.g., every 20th percentile or every 20%. The rings 215 can be interactable or selectable elements in the graphical user interface 175. For example, a user can interact with individual rings 215, select individual rings 215, or activate individual rings 215 to turn the rings on or off to create a set of thresholds or range of thresholds within which the metrics 140 should stay (e.g., the threshold can be values corresponding to the points where the ring 215 intersects with the axes 205). If the values of the metrics 140 satisfy a threshold set through activating one of the rings 215, the computing system 110 can generate, raise, or produce an alert or alarm. An outermost ring 215 can indicate a maximum value for the metrics 140.

The interface manager 160 can generate the radar GUI 190 to indicate points 235 on the axes 205. The interface manager 160 can plot a point 235 on each axes 205 based on a value of the metric 140 for the corresponding metric type of the axes 205. For example, a point 235 representing a value of an energy pedal count metric 140 can be plotted by the interface manager 160 on an axes 205 for the energy pedal count type. A point 235 representing a value of a total instrument path length metric 140 can be plotted by the interface manager 160 on an axes 205 for the total instrument path length type. The interface manager 160 can generate the radar GUI 190 to include segments 230. The segments 230 can be lines, linear segments, curved segments, bent segments between the points 235. The segments 230 can connect points 235 on neighboring axes 205, e.g., axes 205 that are next to each other or where there is no intervening axes 205 between the two axes 205. The interface manager 160 can cause the graphical user interface 175 to plot segments 230 between the points 235 on neighboring axes 205 of the axes 205. The interface manager 160 can plot the lines or segments 230 between points 235 on the axes 205 of the radar GUI 190 to illustrate the metrics 140 for a particular medical session as a shape.

The interface manager 160 can generate shapes 220 or 225. The shapes 220 and 225 can represent a percentile or percentage range for a group of medical procedures. The computing system 110 can receive data streams 115 for multiple different medical procedures, and generate metrics 140 for the different medical procedures. With the metrics 140 generated for the different medical procedures, the computing system 110 can identify or generate at least one percentile for the metrics 140. The interface manager 160 can generate data to cause the graphical user interface 175 to display the radar plot 190 including the at least one percentile of the three or more metrics 140. For example, the computing system 110 can identify metrics 140 for various metric types for a group of medical procedures. For the shape 220, the group of medical procedures can be a set of historical medical procedures for a particular operator of the robotic medical system 105 of a particular type. For the shape 225, the group of medical procedures can be a set of historical medical procedures for operations of the robotic medical system 105 of a particular type for a hospital. The medical procedures can be medical procedures with successful outcomes, the medical procedures can be medical procedures with unsuccessful outcomes, the medical procedures can be medical procedures with outcomes that are both successful and unsuccessful.

The shapes 220 and 225 can include average percentiles for their respective groups. For example, the shapes 220 and 225 can include shading that extends from a twenty fifth percentile to a seventy fifth percentile. For example, for the shape 220, the range can indicate twenty fifth percentile to seventy fifth percentiles of the metrics 140 for historical medical procedures for a particular surgeon. For example, for the shape 225, the range can indicate twenty fifth percentile to seventy fifth percentile of metrics 140 for expert surgeons for a hospital reference. The shapes 220 and 225 can be displayed on the same or different radar plots 190. The interface manager 160 can plot a range of metrics 140 for a group of medical sessions and overlay the metrics 140 for the current medical session onto the radar GUI 190 or the ranges to illustrate the performance of the particular medical session to the group of medical sessions.

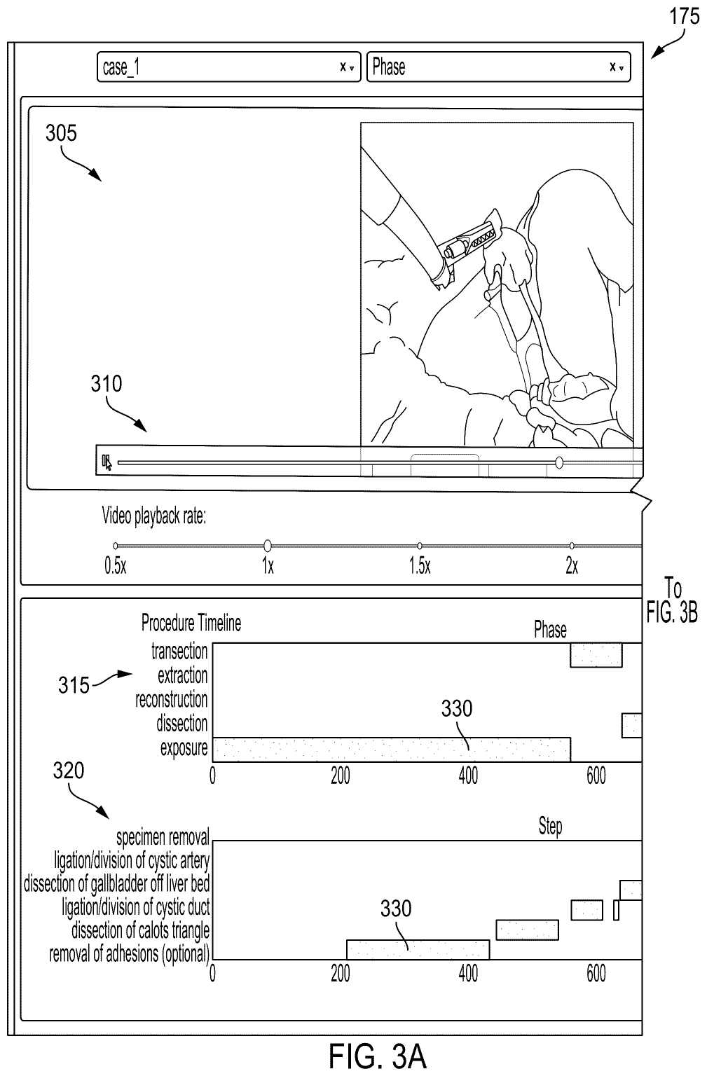

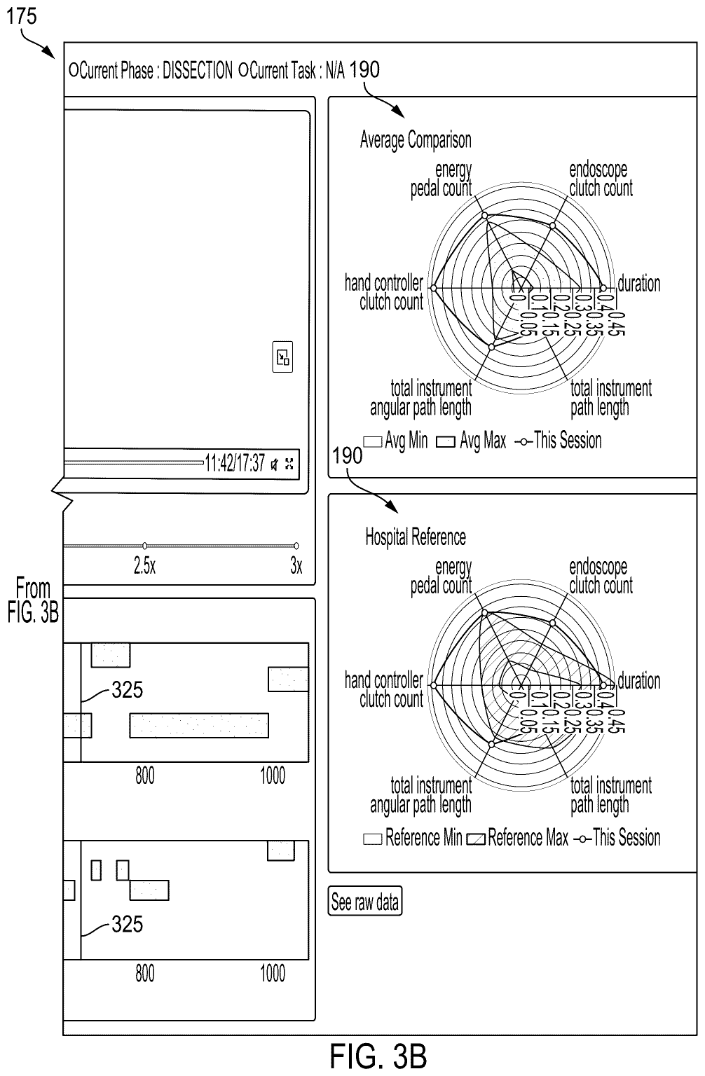

Referring now to FIGS. 3A-B, among others, a graphical user interface 175 including radar charts 190 that correspond to segments of a video 170 of a medical procedure is shown. The graphical user interface 175 can include a video display 305. The video display 305 can display the video 170 and play the video 170 to a user via the graphical user interface 175. The video display 305 can include a timeline 310. The timeline 310 can track the frame or point in the video 170 that is being displayed in the video display 305. A user can provide input via the timeline 310 to scan, seek, or move the video 170 forward or backwards.

The graphical user interface 175 can include procedure timelines 315 and 320. The timeline 315 can indicate phases of the medical procedure. The timeline 320 can indicate steps of the phases of the medical procedure. The timeline 315 can display a plot of the various phases of the medical procedure (e.g., transection, extraction, reconstruction, dissection, or exposure) over time for the video 170. The timeline 320 can display a plot of the various steps of the medical procedure (e.g., specimen removal, litigation/division of cystic artery, or dissection of gallbladder of liver off liver bed) over time for the video 170.

The timelines 315 and 320 can indicate phases or steps of the medical procedure with blocks 330 in a chart. Timelines 315 and 320 can be configured such that a left most side of the timeline 315 indicates an earliest or beginning time in the video 170 while a right most side of the timeline 315 indicates a latest or ending time in the video 170. The blocks 330 can include a left end representing a starting time of the particular phase or step, and a right end representing an ending time of the particular phase or step. A vertical element 325, such as a vertical line, can indicate a current point that the video 170 is being viewed in the video display 305.

The user can scan or navigate through the video 170 the timeline 310, the timeline 315, or the timeline 320. The user can navigate the video 170 to a particular phase or step of the medical procedure. For example, the interface manager 160 can receive the segment selection 180 via the timeline 310, the timeline 315, or the timeline 320. Responsive to receiving the segment selection 180, the interface manager 160 can navigate the video 170 to the requested segment. The interface manager 160 can generate data to cause the graphical user interface 175 to display a portion of the video 170 corresponding to the segment selection 180.

The interface manager 160 can generate data to cause the graphical user interface 175 to cause at least one radar plot 190 of the graphical user interface 175 to display the metrics 140 corresponding to the segment selected by the user. The interface manager 160 can cause the radar charts 190 to plot metrics 140 corresponding to the particular segment, such as the particular phase or particular step of the medical procedure. In this regard, as different segments of the video 170 are selected, or as the video 170 plays through different segments, the radar GUI 190 can update to display metrics 140 for the segment currently being viewed in the graphical user interface 175. In some implementations, the computing system 110 can detect whether at least a predefined number of historical videos including the particular type of selected segment (e.g., phase 130 or step 135 selected by the user) are stored in the data storage 120. For example, if a first type of phase 130 is selected, such as transection, the computing system 110 can determine whether at least a predefined number or threshold number of videos 170 are stored in the data storage 120 that include transection. Responsive to a detection that the threshold number of videos 170 are stored by the data storage 120, the computing system 110 can generate historical average metrics 140 of a variety of types for the selected segment, percentiles for the metrics 140 for the selected segment 180, or percentages for the metrics 140 of the selected segment. Responsive to a detection that the threshold number of videos 170 are not stored in the data storage 120, the computing system 110 may not generate the radar GUI 190 or may not display the percentiles of the metrics 140 in the radar GUI 190.

The interface manager 160 can cause the graphical user interface 175 to include a first radar GUI 190 and a second radar GUI 190. The interface manager 160 can cause the graphical user interface 175 to include a first radar GUI 190 generated by the individual chart generator 150. The interface manager 160 can cause the graphical user interface 175 to include a second radar GUI 190 generated by the group chart generator 155. The first radar GUI 190 can be a plot for an average comparison between the metrics 140 of the particular medical session and a group of other medical sessions for a single surgeon or operator of the robotic medical system 105. For example, the first radar GUI 190 can plot the metrics 140 of the instant medical procedure over a percentile or percentage range for metrics 140 of other medical procedures of the same operator of the robotic medical system 105.

The second radar GUI 190 can be a plot for an average comparison between the metrics 140 of the particular medical session and a group of other medical sessions for a group of surgeons or operators of the robotic medical system 105. The group could be all surgeons or operators at a particular hospital, surgeons at a particular hospital that perform a particular type of surgery, or surgeons of a particular medical practice. For example, the first radar GUI 190 can plot the metrics 140 of the instant medical procedure over a percentile or percentage range for metrics 140 of other medical procedures of the same operator of the robotic medical system 105.

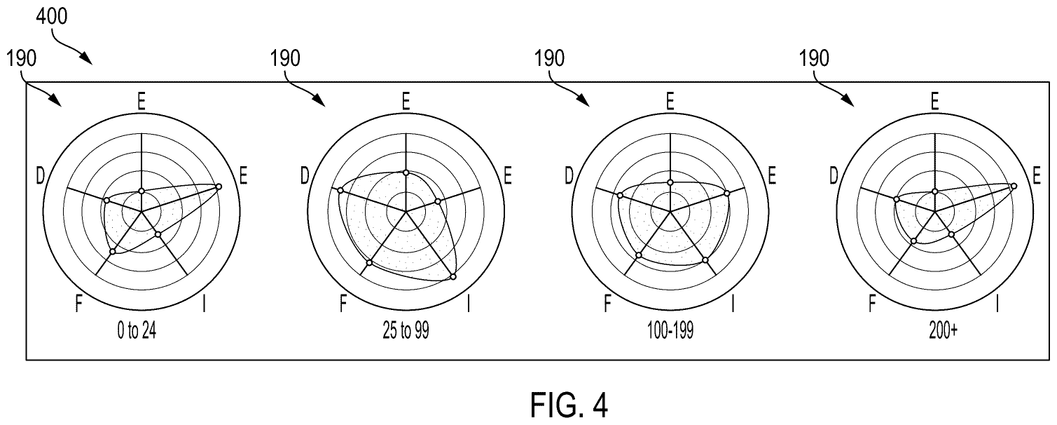

Referring now to FIG. 4, among others, a graphical user interface 400 including comparisons between early and late stage cases is shown. The graphical user interface 400 can include a set of radar charts 190 that are generated for different groups of medical procedures. Each radar GUI 190 can be generated for different stage of a diagnosis or case. For example, the radar charts 190 can be generated by the computing system 110 for early stage cases, middle stage cases, or late stage cases. The radar charts 190 can each indicate a length of time between a diagnosis and the medical procedure. In some cases, the computing system can generate a radar graphical user interface in which the set of radar charts are stacked together on the same radar graphical user interface. The stacked radar charts can illustrate a trend or change in performance during a medical procedures or over multiple medical procedures.

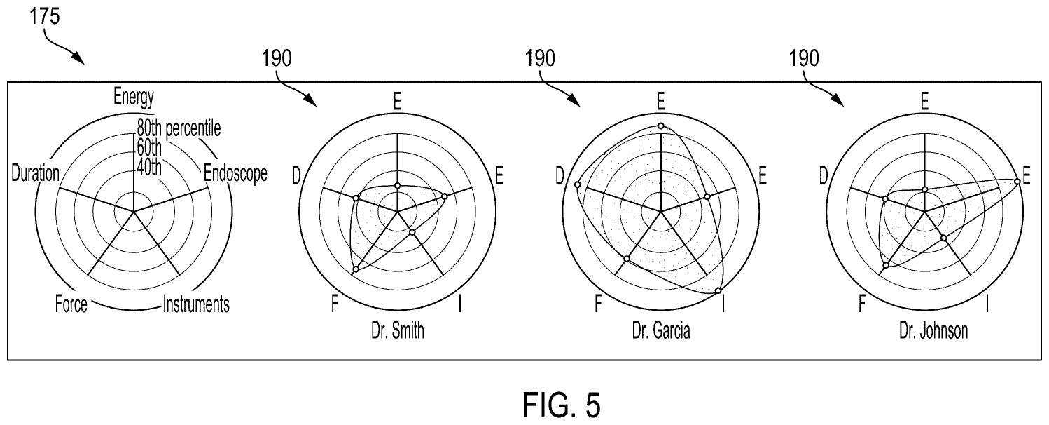

Referring now to FIG. 5, among others, a graphical user interface 175 including radar charts 190 of performance indicators of different medical practitioners is shown. Each radar GUI 190 can be generated using data streams 115 corresponding to one medical practitioner. For example, a first radar GUI 190 can be generated with data streams 115 produced during medical sessions performed by a first medical practitioner (e.g., where the first medical practitioner operated the robotic medical system 105 to perform a procedure). A second radar GUI 190 can be generated with data streams 115 produced during medical sessions performed by a second medical practitioner (e.g., where the second medical practitioner operated the robotic medical system 105 to perform a procedure).

The computing system 110 can receive data streams 115 for multiple different medical practitioners that operated the robotic medical system 105 to perform a medical procedure. For example, the computing system 110 can receive first data streams 115 for first medical procedures performed by a first medical practitioner with the robotic medical system 105. The computing system 110 can receive second data streams 115 for second medical procedures performed by a second medical practitioner with the robotic medical system 105. Each data stream 115 received by the computing system 110 can be labeled or tagged with an indication of the name or identifier of the medical practitioner and stored in the data storage 120. The computing system 110 can generate metrics 140 for the data streams 115 of the various medical practitioners. The computing system 110 can generate metrics 140 for each medical procedure. For example, the computing system 110 can generate metrics 140 for the medical procedures performed by the first medical practitioner using the first data streams 115. The computing system 110 can generate metrics 140 for the medical procedures performed by the second medical practitioner using the second data streams 115.

The computing system 110 can collect or aggregate the metrics 140 together based on medical practitioner. For example, a first set of metrics 140 can be aggregated or grouped together for a first medical practitioner, and a second set of metrics 140 can be aggregated or grouped together for a second medical practitioner. The computing system 110 can generate average or median values for the metrics 140 for each metric type for each medical practitioner. For example, from the first set of metrics 140, the computing system 110 can determine an average metric 140 for each metric type for the first medical practitioner. From the second set of metrics 140, the computing system 110 can determine an average metric 140 for each metric type for the second medical practitioner. The computing system 110 can generate a radar GUI 190 based on the average metrics 140 for each medical practitioner. The radar charts 190 can be displayed together within the graphical user interface 175, along with a label or name of the corresponding medical practitioner.

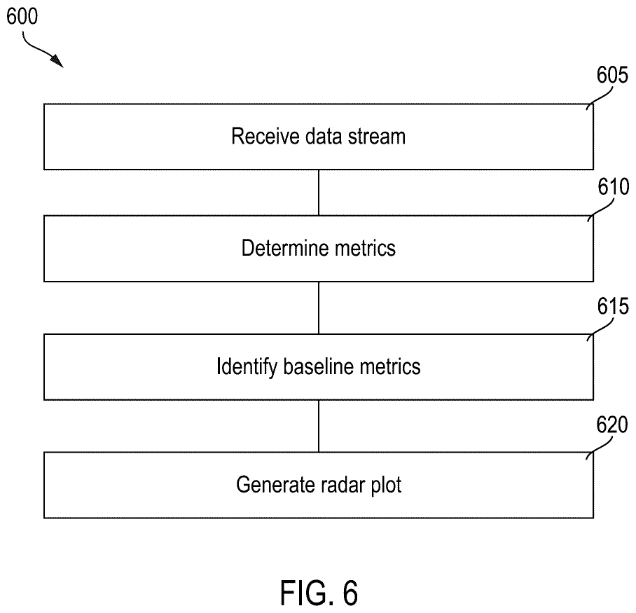

Referring now to FIG. 6, among others, a method 600 of generating a radar GUI 190 including performance indicators of a robotic medical system 105 is shown. At least a portion of the method 600 can be performed by the computing system 110. At least a portion of the method 600 can be performed by the robotic medical system 105. At least a portion of the method 600 can be performed by the client device 165. The method 600 can include receiving a data stream. The method 600 can include determining metrics. The method 600 can include identifying baseline metrics. The method 600 can include generating a radar plot.

At ACT 605, the method 600 can include receiving, by the computing system 110, a data stream 115. The method 600 can include receiving the data stream 115 from the robotic medical system 105. The method 600 can include connecting with the robotic medical system 105 via a network, via an communication channel, or via a bus. The robotic medical system 105 can transmit or send the data stream 115 to the computing system 110 via the connection between the robotic medical system 105 and the computing system 110. The computing system 110 can receive the data stream 115 from the robotic medical system 105 via the connection between the robotic medical system 105 and the computing system 110. The method 600 can include storing the data stream 115 in data storage 120 of the computing system 110.

At ACT 610, the method 600 can include determining, by the computing system 110, metrics 140. The method 600 can include determining, by the computing system 110, the metrics 140 using the data stream 115. The method 600 can compute, generate, or produce the metrics 140 from the metrics 140. For example, the method 600 can include storing at least one equation, relationship, or function. The method 600 can include executing, by the computing system 110, the equation, relationship, or function with the data stream 115 to generate the metrics 140. The method 600 can include generating, by the computing system 110, the metrics 140 for a variety of different metric types. For example, the method 600 can include generating, by the computing system 110, metrics 140 for a first metric type and generating, by the computing system 110, metrics 140 for a second metric type. The method 600 can include generating the various metrics 140 for the various metric types for various segments of the video 170 or the medical procedure. For example, the method 600 can include generating, by the computing system 110, metrics 140 for a phase 130 or step 135 of the medical procedure.

The method 600 can include generating, by the computing system 110, metrics for a smallest segment. The method 600 can include aggregating, by the computing system 110, the metrics 140 into larger segments that the smaller segments make up. For example, the method 600 can include aggregating metrics 140 for the steps 135 for a particular phase 130 into a metric 140 for the phase 130. The method 600 can include aggregating, by the computing system, the metrics 140 for the phases 130 into aggregate metrics 140 for the entire video 170 or medical procedure.

At ACT 615, the method 600 can include identifying, by the computing system 110, baseline metrics 140. The method 600 can include identifying, by the computing system 110, historical videos 170 of medical procedures or medical procedures directly. The method 600 can include generating, by the computing system 110, a baseline metric 140 with the metrics 140 of the metrics 140 of the identified medical procedures. The method 600 can include generating average metrics 140 using the metrics 140 of the identified medical procedures. The method 600 can include generating an average metric 140 for each of multiple metric types. The baseline metrics 140 can be generated for a specific medical practitioner with metrics 140 generated from medical procedures performed by the specific medical practitioner. The baseline metrics 140 can be generated for a specific group medical practitioner with metrics 140 generated from medical procedures performed by the specific group of medical practitioners.

At ACT 620, the method 600 can include generating, by the computing system 110, a radar plot 190. The method 600 can include normalizing, by the computing system 110, the metrics 140 for a particular medical procedure with the baseline metrics 140 identified at ACT 615. The resulting normalized metrics 140 can all share the same numeric range, e.g., zero to ten, zero to one hundred, etc. The method 600 can include plotting, by the computing system 110, the normalized metrics 140 on axes 205 of the radar GUI 190. The method 600 can include plotting percentile ranges for the baseline metrics 140, and overlaying the normalized metrics 140 over the percentile ranges. For example, the method 600 can include generating the radar plot 190 to include shapes 220 or 225 that represent percentile ranges for a particular medical practitioner or a group of medical practitioners.

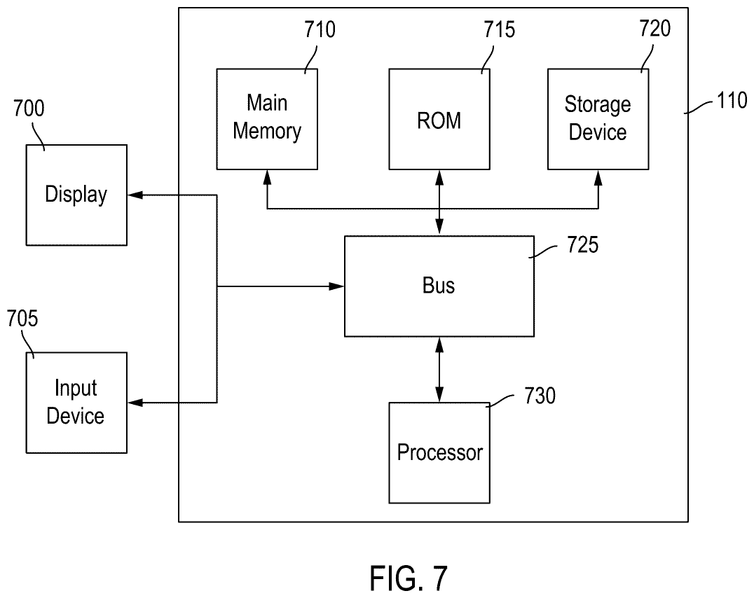

Referring now to FIG. 7, among others, an example block diagram of a computing system 110 is shown. The computing system 110 can include or be used to implement a data processing system or its components. The architecture described in FIG. 7 can be used to implement the computing system 110, the robotic medical system 105, or the client device 165. The computing system 110 can include at least one bus 725 or other communication component for communicating information and at least one processor 730 or processing circuit coupled to the bus 725 for processing information. The computing system 110 can include one or more processors 730 or processing circuits coupled to the bus 725 for processing information. The computing system 110 can include at least one main memory 710, such as a random access memory (RAM) or other dynamic storage device, coupled to the bus 725 for storing information, and instructions to be executed by the processor 730. The main memory 710 can be used for storing information during execution of instructions by the processor 730. The computing system 110 can further include at least one read only memory (ROM) 715 or other static storage device coupled to the bus 725 for storing static information and instructions for the processor 730. A storage device 720, such as a solid state device, magnetic disk or optical disk, can be coupled to the bus 725 to persistently store information and instructions.

The computing system 110 can be coupled via the bus 725 to a display 700, such as a liquid crystal display, or active matrix display. The display 700 can display information to a user. An input device 705, such as a keyboard or voice interface can be coupled to the bus 725 for communicating information and commands to the processor 730. The input device 705 can include a touch screen of the display 700. The input device 705 can include a cursor control, such as a mouse, a trackball, or cursor direction keys, for communicating direction information and command selections to the processor 730 and for controlling cursor movement on the display 700.

The processes, systems and methods described herein can be implemented by the computing system 110 in response to the processor 730 executing an arrangement of instructions contained in main memory 710. Such instructions can be read into main memory 710 from another computer-readable medium, such as the storage device 720. Execution of the arrangement of instructions contained in main memory 710 causes the computing system 110 to perform the illustrative processes described herein. One or more processors in a multi-processing arrangement can be employed to execute the instructions contained in main memory 710. Hard-wired circuitry can be used in place of or in combination with software instructions together with the systems and methods described herein. Systems and methods described herein are not limited to any specific combination of hardware circuitry and software.

Although an example computing system has been described in FIG. 7, the subject matter including the operations described in this specification can be implemented in other types of digital electronic circuitry, or in computer software, firmware, or hardware, including the structures disclosed in this specification and their structural equivalents, or in combinations of one or more of them.

Some of the description herein emphasizes the structural independence of the aspects of the system components or groupings of operations and responsibilities of these system components. Other groupings that execute similar overall operations are within the scope of the present application. Modules can be implemented in hardware or as computer instructions on a non-transient computer readable storage medium, and modules can be distributed across various hardware or computer based components.

The systems described above can provide multiple ones of any or each of those components and these components can be provided on either a standalone system or on multiple instantiations in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C #, PROLOG, Python, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices including cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. ACTs, elements and features discussed in connection with one implementation are not intended to be excluded from a similar role in other implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any ACT or element being based on any information, act or element may include implementations where the act or element is based at least in part on any information, act, or element.

Any implementation disclosed herein may be combined with any other implementation or example, and references to “an implementation,” “some implementations,” “one implementation” or the like are not necessarily mutually exclusive and are intended to indicate that a particular feature, structure, or characteristic described in connection with the implementation may be included in at least one implementation or example. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

What is claimed is:

1. A system, comprising:
one or more processors, coupled with memory, to:
receive, from a robotic medical system, a data stream of a medical procedure performed by the robotic medical system;
determine, using at least the data stream, a plurality of metrics corresponding to a plurality of types, wherein the plurality of metrics are indicative of performance of the medical procedure with the robotic medical system;
identify a plurality of baseline metrics corresponding to the plurality of types; and
generate data to cause a graphical user interface to display a radar plot comprising the plurality of metrics normalized based on the corresponding plurality of baseline metrics.

2. The system of claim 1, comprising:
the one or more processors to:
receive the data stream from the robotic medical system during the medical procedure;
generate the data to cause the graphical user interface to display the radar plot during the medical procedure; and
generate an alarm during the medical procedure responsive to at least one of the plurality of metrics satisfying a threshold.

3. The system of claim 1, comprising:
the one or more processors to:
generate the data to cause the graphical user interface to display the radar plot comprising three or more axes on a single plane to display three or more metrics of the plurality of metrics,
the three or more axes extending outwards from a single point representing a first value of the three or more metrics to second points representing second values of the three or more metrics, the second values greater than the first value.

4. The system of claim 1, comprising:
the one or more processors to:
receive a video of the medical procedure from the robotic medical system;
receive, via the graphical user interface, a selection of a segment of a plurality of segments of the video of the medical procedure; and
generate the data to cause the graphical user interface to display a portion of the video corresponding to the segment and the radar plot to display the plurality of metrics corresponding to the segment.

5. The system of claim 1, comprising:
the one or more processors to:
receive a video of the medical procedure from the robotic medical system;
cause the graphical user interface to include an element to play the video;
cause the graphical user interface to include a timeline including a plurality of segments;
receive, via the element or the timeline, a scan of the video to a point corresponding to a segment of the plurality of segments of the medical procedure;
select the segment from the plurality of segments responsive to receiving the scan to the point; and
generate the data to cause the graphical user interface to display a portion of the video corresponding to the segment and the radar plot to display the plurality of metrics corresponding to the segment.

6. The system of claim 1, comprising:
the one or more processors to:
receive a selection to display the plurality of metrics for an entirety of the medical procedure;
generate the plurality of metrics for the entirety of the medical procedure using the data stream; and
generate the data to cause the graphical user interface to display the radar plot to display the plurality of metrics for the entirety of the medical procedure.

7. The system of claim 1, comprising:
the one or more processors to:
receive, via the graphical user interface, a selection of a segment of a plurality of segments of the medical procedure, wherein the segment is a phase of the medical procedure, the phase comprising at least one of:
transection of an anatomical structure of a patient;
extraction of the anatomical structure;
reconstruction of the anatomical structure after opening the anatomical structure;
dissection of the anatomical structure; or
exposure of the anatomical structure; and
generate the data to cause the graphical user interface to display the radar plot to display the plurality of metrics for the segment.

8. The system of claim 1, comprising:
the one or more processors to:
receive, via the graphical user interface, a selection of a step of a plurality of steps of a particular phase of a plurality of phases of the medical procedure; and
generate the data to cause the graphical user interface to display the radar plot to display the plurality of metrics for the step.

9. The system of claim 1, comprising:
the one or more processors to:
generate, using at least the data stream, the plurality of metrics comprising at least one of:
an amount of energy consumed by the robotic medical system;
a total duration of a segment of the medical procedure;
a total linear distance an instrument of the robotic medical system travelled during the segment;
a total angular distance the instrument of the robotic medical system travelled during the segment;
a total number of operations of a clutch or brake for the instrument of the robotic medical system; or
a total number of operations of a clutch or brake for an endoscope of the robotic medical system.

10. The system of claim 1, comprising:
the one or more processors to:
cause the graphical user interface to plot points on three or more axes of the radar plot based on values of the plurality of metrics; and
cause the graphical user interface to plot segments between the points on neighboring axes of the three or more axes.

11. The system of claim 1, comprising:
the one or more processors to:
receive a plurality of data streams for a plurality of medical procedures;
generate the plurality of metrics for the plurality of medical procedures;
determine at least one percentile of the plurality of metrics; and
generate the data to cause the graphical user interface to display the radar plot comprising three or more axes on a single plane to display three or more metrics of the plurality of metrics and the at least one percentile of the three or more metrics.

12. The system of claim 1, comprising:
the one or more processors to:
receive a first plurality of data streams for a first plurality of medical procedures performed by a first medical practitioner with the robotic medical system;
generate, using at least the first plurality of data streams, a plurality of first metrics for the first medical practitioner;
receive a second plurality of data streams for a second plurality of medical procedures performed by a second medical practitioner with the robotic medical system;
generate, using at least the second plurality of data streams, a plurality of second metrics for the first medical practitioner; and
generate the data to cause the graphical user interface to include:
a first radar plot to display the plurality of first metrics for the first medical practitioner; and
a second radar plot to display the plurality of second metrics for the second medical practitioner.

13. The system of claim 1, comprising:
the one or more processors to:
receive, via the graphical user interface, a shape drawn on the radar plot, the shape identifying points on three or more axes of the radar plot;
detect that a value of one metric of the plurality of metrics is greater than or less than a value corresponding to one point of the points drawn on an axis of the one metric; and
generate an alarm responsive to a detection that the value of the one metric is greater than or less than the value corresponding to the one point.

14. The system of claim 1, comprising:
the one or more processors to:
receive, via the graphical user interface, a shape drawn on the radar plot, the shape defined by points on three or more axes of the radar plot;
search a plurality of medical procedures using the shape drawn on the radar plot to identify a second medical procedure associated with the plurality of metrics that conforms to the shape; and
cause the graphical user interface to display a video of the second medical procedure.

15. A method, comprising:
receiving, by one or more processors, coupled with memory, from a robotic medical system, a data stream of a medical procedure performed by the robotic medical system;
determining, by the one or more processors, using at least the data stream, a plurality of metrics corresponding to a plurality of types, wherein the plurality of metrics are indicative of performance of the medical procedure with the robotic medical system;
identifying, by the one or more processors, a plurality of baseline metrics corresponding to the plurality of types; and
generating, by the one or more processors, data to cause a graphical user interface to display a radar plot comprising the plurality of metrics normalized based on the corresponding plurality of baseline metrics.

16. The method of claim 15, comprising:
receiving, by the one or more processors, the data stream from the robotic medical system during the medical procedure;
generating, by the one or more processors, the data to cause the graphical user interface to display the radar plot during the medical procedure; and
generating, by the one or more processors, an alarm during the medical procedure responsive to at least one of the plurality of metrics satisfying a threshold.

17. The method of claim 15, comprising:
receiving, by the one or more processors, a video of the medical procedure from the robotic medical system;
causing, by the one or more processors, the graphical user interface to include an element to play the video;
causing, by the one or more processors, the graphical user interface to include a timeline including a plurality of segments;
receiving, by the one or more processors, via the element or the timeline, a scan of the video to a point corresponding to a segment of the plurality of segments of the medical procedure;
selecting, by the one or more processors, the segment from the plurality of segments responsive to receiving the scan to the point; and
generating, by the one or more processors, the data to cause the graphical user interface to display a portion of the video corresponding to the segment and the radar plot to display the plurality of metrics corresponding to the segment.

18. The method of claim 15, comprising:
receiving, by the one or more processors, a selection to display the plurality of metrics for an entirety of the medical procedure;
generating, by the one or more processors, the plurality of metrics for the entirety of the medical procedure using the data stream; and
generating, by the one or more processors the data to cause the graphical user interface to display the radar plot to display the plurality of metrics for the entirety of the medical procedure.

19. One or more storage media storing instructions thereon, that, when executed by one or more processors, cause the one or more processors to:
receive, from a robotic medical system, a data stream of a medical procedure performed by the robotic medical system;
determine, using at least the data stream, a plurality of metrics corresponding to a plurality of types, wherein the plurality of metrics are indicative of performance of the medical procedure with the robotic medical system;
identify a plurality of baseline metrics corresponding to the plurality of types; and
generate data to cause a graphical user interface to display a radar plot comprising the plurality of metrics normalized based on the corresponding plurality of baseline metrics.

20. The one or more storage media of claim 19, wherein the instructions cause the one or more processors to:
receive a video of the medical procedure from the robotic medical system;
cause the graphical user interface to include an element to play the video;
cause the graphical user interface to include a timeline including a plurality of segments;
receive via the element or the timeline, a scan of the video to a point corresponding to a segment of the plurality of segments of the medical procedure;
select the segment from the plurality of segments responsive to receiving the scan to the point; and
generate the data to cause the graphical user interface to display a portion of the video corresponding to the segment and the radar plot to display the plurality of metrics corresponding to the segment.
